SEQUENCE LISTING (14 sequences)

SEQ ID NO: 1 - DNA, 20 bp, Artificial Sequence
  Mycoplasma bacteria Forward primer
aaatccaggtacgggtgaag

SEQ ID NO: 2 - DNA, 28 bp, Artificial Sequence
  Mycoplasma bacteria Reverse primer
gtcctgatcaatattaagctacagtaaa

SEQ ID NO: 3 - DNA, 25 bp, Artificial Sequence
  Mycoplasma bacteria Forward primer
aaatccaggtacgggtgaagacacc

SEQ ID NO: 4 - DNA, 36 bp, Artificial Sequence
  Mycoplasma bacteria Reverse primer
gtcctgatcaatattaagctacagtaaagcttcacg

SEQ ID NO: 5 - DNA, 29 bp, Artificial Sequence
  Mycoplasma bacteria Forward primer
ggtgaaatccaggtacgggtgaagacacc

SEQ ID NO: 6 - DNA, 39 bp, Artificial Sequence
  Mycoplasma bacteria Reverse primer
gtcctgatcaatattaagctacagtaaagcttcacgggg

SEQ ID NO: 7 - DNA, 31 bp, Artificial Sequence
  Mycoplasma bacteria Forward primer
ggtgaaatccaggtacgggtgaagacacccg

SEQ ID NO: 8 - DNA, 50 bp, Artificial Sequence
  Mycoplasma bacteria Reverse primer
catgataatgtcctgatcaatattaagctacagtaaagcttcacggggtc

SEQ ID NO: 9 - DNA, 39 bp, Artificial Sequence
  Mycoplasma bacteria Forward primer
ggtgaaatccaggtacgggtgaagacacccgttaggcgc

SEQ ID NO: 10 - DNA, 77 bp, Artificial Sequence
  Mycoplasma bacteria Reverse primer
gcatcgattgctcctacctattctctacatgataatgtcctgatcaatattaagctacagtaaagcttcacggggtc

SEQ ID NO: 11 - DNA, 15 bp, Artificial Sequence
  Mycoplasma bacteria Fluorescent probe
cgggacggaaagacc

SEQ ID NO: 12 - DNA, 31 bp, Artificial Sequence
  Bordetella pertussis Forward primer
atcaagcaccgctttacccgaccttaccgcc

SEQ ID NO: 13 - DNA, 33 bp, Artificial Sequence
  Bordetella pertussis Reverse primer
ttgggagttctggtaggtgtgagcgtaagccca

SEQ ID NO: 14 - DNA, 22 bp, Artificial Sequence
  Bordetella pertussis Fluorescent probe
aatggcaaggccgaacgcttca

(19) United States
(12) Patent Application Publication
(10) Pub. No.: US 2018/0030510 A1
(43) Pub. Date: Feb. 1, 2018

(54) NUCLEIC ACID AMPLIFICATION REACTION METHOD AND NUCLEIC ACID AMPLIFICATION REACTION REAGENT

(71) Applicant: Seiko Epson Corporation, Tokyo (JP)

(72) Inventors: Kotaro IDEGAMI, Chino (JP); Masayuki UEHARA, Matsumoto (JP); Masato HANAMURA, Shiojiri (JP)

(21) Appl. No.: 15/652,718

(22) Filed: Jul. 18, 2017

(30) Foreign Application Priority Data
Jul. 29, 2016 (JP) 2016-149490

(51) Int. Cl. C12Q 1/68; G01N 1/40; G01N 27/447

(52) U.S. Cl. CPC C12Q 1/686; C12Q 1/6806; G01N 27/447; C12Q 1/6853; G01N 1/40; G01N 2001/4038; C12Q 1/00

(57) ABSTRACT

A nucleic acid amplification reaction method includes performing thermal cycling for amplifying a nucleic acid for a reaction solution containing a primer and a probe, wherein in the thermal cycling, the time per cycle of the thermal cycling is 9 seconds or less, and the Tm value of the primer is 70° C. or higher and 80° C. or lower.

### BACKGROUND

### 1. Technical Field

The present invention relates to a nucleic acid amplification reaction method and a nucleic acid amplification reaction reagent.

### 2. Related Art

In recent years, due to the development of technologies utilizing genes, medical treatments utilizing genes such as gene diagnosis or gene therapy have been drawing attention. In addition, many methods using genes in determination of breed varieties or breed improvement have also been developed in agriculture and livestock industries. As technologies for utilizing genes, technologies such as a PCR (Polymerase Chain Reaction) method are widely used. Nowadays, the PCR method has become an indispensable technology for elucidation of information on biological materials.

The PCR method is a method of amplifying a target nucleic acid by performing thermal cycling for a solution (reaction solution) containing a nucleic acid to be amplified (target nucleic acid) and a reagent. The thermal cycling is a treatment of periodically subjecting the reaction solution to two or more temperature steps. In the PCR method, a method of performing two- or three-step thermal cycling is generally used.

An increase in PCR speed is a necessary technology for reducing the testing time of a genetic test, and has been much expected in the genetic testing industries.

For example, JP-T-2015-520614 (Patent Document 1) discloses a method in which a polymerase is provided at a concentration of at least 0.5 μM and a primer is provided at a concentration of at least 2 μM, and a cycle is completed in a cycle time of less than 20 seconds per cycle.

However, in the method disclosed in Patent Document 1, a large amount of a polymerase is used, and therefore, the method is not preferred from the viewpoint of cost for testing. As a result of intensive studies, the present inventors found out a method capable of increasing the PCR speed without using a large amount of a polymerase.

### SUMMARY

An advantage of some aspects of the invention is to provide a nucleic acid amplification reaction method capable of increasing the PCR speed. Another advantage of some aspects of the invention is to provide a nucleic acid amplification reaction reagent capable of increasing the PCR speed.

A nucleic acid amplification reaction method according to an aspect of the invention includes performing thermal cycling for amplifying a nucleic acid for a reaction solution containing a primer and a probe, wherein in the thermal cycling, the time per cycle of the thermal cycling is 9 seconds or less, and the Tm value of the primer is 70° C. or higher and 80° C. or lower.

According to such a nucleic acid amplification reaction method, while increasing the PCR speed, amplification of a nonspecific nucleic acid can be more reliably suppressed (see the below-mentioned “3. Experimental Examples” for the details).

In the nucleic acid amplification reaction method according to the aspect of the invention, a heating time for an annealing reaction for the primer may be 6 seconds or less.

According to such a nucleic acid amplification reaction method, while increasing the PCR speed, amplification of a nonspecific nucleic acid can be more reliably suppressed (see the below-mentioned “3. Experimental Examples” for the details).

In the nucleic acid amplification reaction method according to the aspect of the invention, the reaction solution may contain a divalent cation, and the concentration of the divalent cation contained in the reaction solution may be 2 mM or more and 7.5 mM or less.

According to such a nucleic acid amplification reaction method, while accelerating an elongation reaction by a polymerase and increasing the PCR speed, nonspecific amplification is suppressed, and a decrease in yield of a specific amplification product can be suppressed.

In the nucleic acid amplification reaction method according to the aspect of the invention, the reaction solution may contain MgCl₂, the divalent cation may be derived from MgCl₂, and the concentration of MgCl₂ contained in the reaction solution may be 4 mM or more and 7.5 mM or less.

According to such a nucleic acid amplification reaction method, while increasing the PCR speed, nonspecific amplification is suppressed, and a decrease in yield of a specific amplification product can be suppressed.

In the nucleic acid amplification reaction method according to the aspect of the invention, the reaction solution may contain MgSO₄, the divalent cation may be derived from MgSO₄, and the concentration of MgSO₄ contained in the reaction solution may be 2 mM or more and 3 mM or less.

According to such a nucleic acid amplification reaction method, while increasing the PCR speed, nonspecific amplification is suppressed, and a decrease in yield of a specific amplification product can be suppressed.

In such a nucleic acid amplification reaction method, an optimal concentration range of the divalent cation for suppressing nonspecific amplification and suppressing a decrease in yield of a specific amplification product while accelerating an elongation reaction and increasing the PCR speed varies depending on the type of the divalent cation.

In the nucleic acid amplification reaction method according to the aspect of the invention, the Tm value of the primer may be 70° C. or higher and 75° C. or lower.

According to such a nucleic acid amplification reaction method, while increasing the PCR speed, amplification of a nonspecific nucleic acid can be more reliably suppressed.

In the nucleic acid amplification reaction method according to the aspect of the invention, the primer may contain an artificial nucleic acid.

According to such a nucleic acid amplification reaction method, the Tm value of a primer can be made to fall within the range of 70° C. or higher and 80° C. or lower without increasing the number of bases of the primer, and nonspecific adsorption of a primer dimer can be suppressed.

In the nucleic acid amplification reaction method according to the aspect of the invention, in the heating for the annealing reaction for the primer, an elongation reaction may be performed.

According to such a nucleic acid amplification reaction method, the PCR speed can be increased as compared with the case where heating for the annealing reaction for the primer and heating for the elongation reaction are performed separately.

In the nucleic acid amplification reaction method according to the aspect of the invention, the reaction solution may contain a probe, and the probe may be a hydrolysis probe.

According to such a nucleic acid amplification reaction method, in PCR, when the probe is degraded by the polymerase, a quenching effect is cancelled, and a reporter dye emits light, whereby the amplification amount of a nucleic acid can be quantitatively determined.

In the nucleic acid amplification reaction method according to the aspect of the invention, the probe may contain at least one of an artificial nucleic acid and a minor groove binder molecule.

According to such a nucleic acid amplification reaction method, in PCR, the Tm value of the probe can be increased without increasing the number of bases of the probe.

A nucleic acid amplification reaction reagent according to an aspect of the invention is a nucleic acid amplification reaction reagent for amplifying a nucleic acid, and includes a primer, a probe, and MgCl₂, wherein the Tm value of the primer is 70° C. or higher and 80° C. or lower, and when the nucleic acid amplification reaction reagent becomes a reaction solution for performing a nucleic acid amplification reaction, the concentration of MgCl₂ contained in the reaction solution is 4 mM or more and 7.5 mM or less.

According to such a nucleic acid amplification reaction reagent, in PCR, while increasing the PCR speed, amplification of a nonspecific nucleic acid can be suppressed.

A nucleic acid amplification reaction reagent according to an aspect of the invention is a nucleic acid amplification reaction reagent for amplifying a nucleic acid, and includes a primer, a probe, and MgSO₄, wherein the Tm value of the primer is 70° C. or higher and 80° C. or lower, and when the nucleic acid amplification reaction reagent becomes a reaction solution for performing a nucleic acid amplification reaction, the concentration of MgSO₄ contained in the reaction solution is 2 mM or more and 3 mM or less.

According to such a nucleic acid amplification reaction reagent, in PCR, while increasing the PCR speed, amplification of a nonspecific nucleic acid can be suppressed.

In the nucleic acid amplification reaction reagent according to the aspect of the invention, the reagent may be lyophilized.

According to such a nucleic acid amplification reaction reagent, the reagent can be stably stored.

### BRIEF DESCRIPTION OF THE DRAWINGS

The invention will be described with reference to the accompanying drawings, wherein like numbers reference like elements.

FIG. 1 is a graph showing a relationship between a temperature for Taq polymerase and a relative activity efficiency.

FIG. 2 is a flowchart for illustrating a nucleic acid amplification reaction method according to an embodiment.

FIG. 3 is a cross-sectional view schematically showing a thermal cycler for performing thermal cycling for a reaction solution according to an embodiment.

FIG. 4 is a graph showing a relationship between a PCR reaction time and a fluorescence intensity.

FIG. 5 is a graph showing a relationship between a PCR reaction time and a fluorescence intensity.

FIG. 6 shows the results of electrophoresis.

FIG. 7 shows the results of electrophoresis.

FIG. 8 is a graph showing a fluorescence intensity.

FIG. 9 is a graph showing a fluorescence intensity.

### DESCRIPTION OF EXEMPLARY EMBODIMENTS

Hereinafter, preferred embodiments of the invention will be described in detail with reference to the accompanying drawings. Note that the embodiments described below are not intended to unduly limit the content of the invention described in the appended claims. Further, all the configurations described below are not necessarily essential components of the invention.

### 1. Nucleic Acid Amplification Reaction Reagent

First, a nucleic acid amplification reaction reagent according to this embodiment will be described. The nucleic acid amplification reaction reagent is a reagent for amplifying a nucleic acid in PCR. The nucleic acid amplification reaction reagent may be, for example, in a liquid form or may be in a lyophilized state. For example, the nucleic acid amplification reaction reagent in a lyophilized state is fixed in a container (not shown), and a template nucleic acid solution containing a DNA (deoxyribonucleic acid) or an RNA (ribonucleic acid) is introduced into the container so as to bring the template nucleic acid solution and the nucleic acid amplification reaction reagent into contact with each other. The nucleic acid amplification reaction reagent in a lyophilized state is dissolved in the aqueous component of the template nucleic acid solution and incorporated into the template nucleic acid solution so as to become a reaction solution. Therefore, the reaction solution contains the template nucleic acid and the nucleic acid amplification reaction reagent, and thus serves as a place for allowing a nucleic acid amplification reaction to proceed.

The nucleic acid amplification reaction reagent contains primers, a polymerase, a probe, dNTP, and a buffer.

### 1.1. Primer

The primer is designed to anneal to a template nucleic acid (template). The “anneal (annealing)” refers to an action (a phenomenon) in which a primer binds to a DNA. The nucleic acid amplification reaction reagent contains a forward primer which anneals to one template nucleic acid having a single-stranded structure (single-stranded DNA) after a template nucleic acid having a double-stranded structure (double-stranded DNA) is denatured, and a reverse primer which anneals to the other single-stranded DNA. The concentrations of the forward primer and the reverse primer contained in the reaction solution are each, for example, 0.4 μM or more and 6.4 μM or less, preferably 0.8 μM or more and 3.2 μM or less. The concentration of the forward primer and the concentration of the reverse primer contained in the reaction solution are, for example, the same.

The Tm value of the primer (the forward primer and the reverse primer) is 70° C. or higher and 80° C. or lower, preferably 70° C. or higher and 75° C. or lower, more preferably 75° C. According to this, the nucleic acid amplification reaction reagent according to this embodiment can increase the PCR speed (see the below-mentioned “3. Experimental Examples” for the details). The Tm value is an index of the temperature at which a primer anneals to a template nucleic acid, and is a temperature at which 50% of a double-stranded DNA is dissociated into single-stranded DNAs, that is, a melting temperature. If the temperature is not lower than the Tm value, not less than half of the primer anneals to the template nucleic acid. The Tm value of the forward primer and the Tm value of the reverse primer may be the same as or different from each other.

As a calculation method of the Tm value, for example, a nearest neighbor method is exemplified, and the Tm value can be calculated according to the following formula (1).

Tm=1000 ΔH/(−10.8+Δs+R×ln(Ct/4))−273.15+16.6 log[Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primers, and Na⁺ represents the concentration (mol/L) of a monovalent cation contained in the buffer.

The primer may contain an artificial nucleic acid. According to this, the Tm value of the primer can be made to fall within the above range without increasing the number of bases of the primer. When the number of bases of the primer is increased, nonspecific adsorption occurs or the primers form a primer dimer (double strand), and a target nucleic acid cannot be amplified in some cases. As the primer dimer, there are a self-dimer which forms a double strand in one primer, and a cross-dimer which forms a double strand between the forward primer and the reverse primer.

The “artificial nucleic acid” refers to a nucleic acid molecule which can bind to abase of a DNA or an RNA through a hydrogen bond and is other than natural nucleic acid molecules. Examples of the artificial nucleic acid include a 2′,4′-BNA (2′-O,4′-C-methano-bridged nucleic acid, also known as “LNA (Locked Nucleic Acid)”) in which the oxygen atom at the 2′-position of a ribose ring of a nucleic acid is methylene-crosslinked to the carbon atom at the 4′-position. The chemical formula of the LNA is shown in the following formula (2).

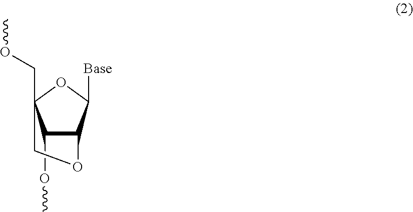

In the formula (2), examples of the base include T (thymine), C (cytosine), G (guanine), and A (adenine), but are not particularly limited. Further, the base may be a base modified by methylation, acetylation, or the like.

The artificial nucleic acid may be an LNA analog obtained by modifying an LNA, and may be specifically 3′-amino-2′,4′-BNA, 2′,4′-BNA^{COC}, or 2′,4′-BNA^{Nc}. Further, an artificial nucleic acid contained in a modified fluorescent probe may be a PNA (Peptide Nucleic Acid), a GNA (Glycol Nucleic Acid), a TNA (Threose Nucleic Acid), or an analog obtained by modifying such a molecule. The number of artificial nucleic acids contained in the probe is not particularly limited, and one probe may contain a plurality of artificial nucleic acids.

In the case where the Tm value of the primer is increased by increasing the number of bases of the primer, by designing the primer so as to be elongated inside the amplification region (so that the primer is elongated on the 5′-end side of the template nucleic acid), the Tm value can be increased without increasing the amplification region of a nucleic acid by the elongation reaction. According to this, the PCR speed can be increased.

### 1.2. Polymerase

The polymerase is not particularly limited, however, examples thereof include a DNA polymerase. The DNA polymerase polymerizes nucleotides complementary to the bases of a template nucleic acid at the end of the primer annealing to the template nucleic acid having a single-stranded structure (single-stranded DNA). The DNA polymerase is preferably a heat-resistant enzyme or an enzyme for PCR, and there are a large number of commercially available products, for example, Taq polymerase, KOD polymerase, Tfi polymerase, Tth polymerase, modified forms thereof, and the like, however, a DNA polymerase capable of performing hot start is preferred. As the polymerase, there are a hydrolysis-type polymerase which degrades a probe by hydrolysis such as Taq polymerase, and a non-hydrolysis-type polymerase which does not degrade a probe by hydrolysis such as KOD polymerase. The KOD polymerase is derived from Thermococcus kodakarensis KOD1 and is a DNA polymerase from the genus Thermococcus. The amount of the polymerase contained in the reaction solution is, for example, 0.5 U or more.

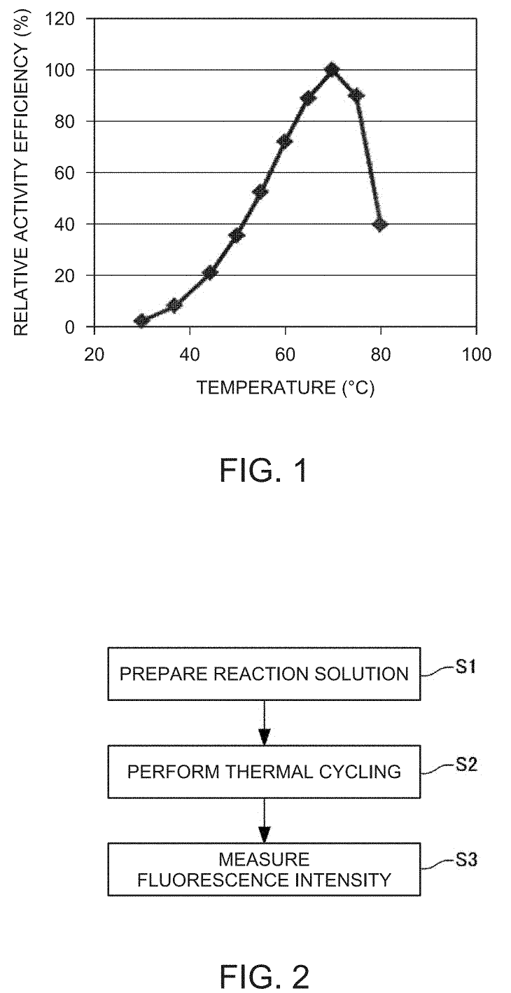

FIG. 1 is a graph showing a relationship between a temperature for Taq polymerase and a relative activity efficiency. The vertical axis represents a relative activity efficiency when the maximum value of the activity efficiency of the polymerase which is reached while changing the temperature is assumed to be 100%. The activity efficiency of the polymerase at each temperature can be obtained by performing a procedure so that dNTP emits light when it is incorporated during the elongation reaction, and measuring the fluorescence intensity (fluorescent brightness) from the dNTP after a predetermined period of time has elapsed. As the fluorescence intensity is higher, the activity efficiency of the polymerase is higher. In the example shown in FIG. 1, the relative activity efficiency of the Taq polymerase reached the maximum when the temperature was around 70° C.

1.3. dNTP

The dNTP refers to a mixture of four types of deoxyribonucleotide triphosphates. That is, the dNTP refers to a mixture of dATP, dCTP, dGTP, and dTTP. The DNA polymerase forms a new DNA by joining dATP, dCTP, dGTP, or dTTP to the end of the primer annealing to the template (an elongation reaction). The concentration of the dNTP contained in the reaction solution is, for example, 0.06 mM or more and 0.75 mM or less, preferably 0.125 mM or more and 0.5 mM or less.

### 1.4. Probe

The probe is a fluorescently labeled probe to be used for quantitatively determining the amplification amount of a nucleic acid. The probe may be a hydrolysis probe containing a reporter dye and a quencher dye. More specifically, the probe is TaqMan (registered trademark) probe. While the hydrolysis probe hybridizes to a single-stranded DNA to form a double-stranded structure, the light emission of a reporter dye is suppressed by a quencher dye (by a quenching effect) which is in close proximity to the reporter dye. However, when the probe is degraded by the exonuclease activity of the polymerase, the quenching effect is cancelled, and therefore, the reporter dye emits light. By this light emission, the amplification amount of a nucleic acid can be quantitatively determined. The “hybridization” refers to a phenomenon in which a probe binds to a DNA. The concentration of the probe contained in the reaction solution is 0.5 μM or more and 2.4 μM or less, preferably 0.5 μM or more and 1.8 μM or less.

The probe may be a non-hydrolysis probe other than the hydrolysis probe. Specifically, the probe may be a Q (Quenching) probe utilizing a fluorescence-quenching phenomenon. The Q probe emits light in a state where it does not hybridize to a single-stranded DNA, and quenches the light when it hybridizes to a single-stranded DNA. By this difference in the emission intensity, the amplification amount of a nucleic acid can be quantitatively determined. In the case where the Q probe is used as the probe, KOD polymerase is used as the polymerase. The elongation reaction rate of KOD polymerase is larger than that of Taq polymerase, and therefore, KOD polymerase can increase the thermal cycling speed.

In the case where the probe is a non-hydrolysis probe, it is not necessary to degrade the probe in the elongation reaction, and therefore, there is no need to provide an amplification region to which the probe anneals between the forward primer and the reverse primer. According to this, it becomes possible to design the amplification region narrower than in a hydrolysis-type system. Since the amplification region becomes narrower, the annealing time can be reduced, and thus, the thermal cycling speed can be increased.

The probe may contain at least one of an artificial nucleic acid and a minor groove binder molecule. According to this configuration, the Tm value of the probe can be increased without increasing the number of bases of the probe. When the number of bases of the probe is increased, the elongation reaction time is increased for degrading the probe (a region of a nucleic acid to be amplified is increased in the elongation reaction), and therefore, it is sometimes difficult to increase the PCR speed. As the artificial nucleic acid, the above-mentioned artificial nucleic acid can be used.

In this embodiment, the Tm value of the primer is 70° C. or higher, which is high, and basically, the number of bases is large (the base length is long). In general, as the number of bases is increased, the occurrence ratio of nonspecific amplification is also increased, and the specificity of PCR itself is decreased. However, in this embodiment, the specificity can be increased by the probe.

In this embodiment, in order to detect a nucleic acid amplification reaction, a probe is used, however, in order to increase the PCR speed, an intercalator is used in place of the probe in some cases. This intercalator method can eliminate a step of hydrolyzing the probe, and therefore, it can be expected to reduce the amplification reaction time. However, in the intercalator method, nonspecific annealing which is one of the problems of the PCR reaction occurs, and due to this, also in the case where a nucleic acid other than the target nucleic acid is amplified, it is detected as a fluorescent brightness. By increasing the speed of the reaction to reduce the reaction time, the above-mentioned nonspecific amplification can be suppressed to some extent, but cannot completely suppressed under the condition that nonspecific amplification is likely to occur, for example, in the case where a human genome DNA is mixed in the reaction solution, and as a result, the specificity of the detection system is decreased. On the other hand, by using the probe, even under the reaction condition that the above-mentioned nonspecific amplification is likely to occur, and even if nonspecific amplification occurs in the amplification step, since the probe has sequence specificity, the specificity of the detection system is ensured.

### 1.5. Buffer

The buffer is, for example, a buffer agent containing a salt. Examples of the salt contained in the buffer include salts such as Tris, HEPES, PIPES, and phosphates. By using such a salt, the pH of the buffer can be adjusted.

The buffer contains a divalent cation. Examples of the divalent cation include Mn, Co, and Mg. In the case where the nucleic acid amplification reaction reagent becomes a reaction solution for performing a nucleic acid amplification reaction, the concentration of the divalent cation contained in the reaction solution is 2 mM or more and 7.5 mM or less. By setting the concentration of the divalent cation to 2 mM or more, the elongation reaction by the polymerase is accelerated, and the PCR speed can be increased (specifically, the time per cycle of the thermal cycling can be reduced to 9 seconds or less). By setting the concentration of the divalent cation to 7.5 mM or less, nonspecific amplification is suppressed, and a decrease in yield of a specific amplification product can be suppressed.

Specifically, the buffer contains a divalent cationic compound, KCl, and Tris. More specifically, the buffer contains MgCl₂, and the divalent cation is derived from MgCl₂. That is, the divalent cation is produced by ionization of MgCl₂. In the case where the divalent cation is derived from MgCl₂, the concentration of Mg²⁺ is attributed to the activity of the polymerase. In the case where the nucleic acid amplification reaction reagent becomes a reaction solution for performing a nucleic acid amplification reaction, the concentration of MgCl₂ contained in the reaction solution is 4 mM or more and 7.5 mM or less, preferably 5 mM or more and mM or less, more preferably 5 mM. By setting the concentration of MgCl₂ to 4 mM or more, the elongation reaction by the polymerase is accelerated, and the PCR speed can be increased. By setting the concentration of MgCl₂ to 7.5 mM or less, nonspecific amplification is suppressed, and a decrease in yield of a specific amplification product can be suppressed. When the nucleic acid amplification reaction reagent is in a lyophilized state, the nucleic acid amplification reaction reagent is in a solid state, and contains MgCl₂, KCl, Tris, and an excipient such as trehalose.

The divalent cationic compound may be derived from MgSO₄. In this case, the buffer contains MgSO₄ in place of MgCl₂, and in the case where the nucleic acid amplification reaction reagent becomes a reaction solution for performing a nucleic acid amplification reaction, the concentration of MgSO₄ contained in the reaction solution is 2 mM or more and 3 mM or less, more preferably 2 mM. By setting the concentration of MgSO₄ to 2 mM or more, the elongation reaction by the polymerase is accelerated, and the PCR speed can be increased. By setting the concentration of MgSO₄ to 3 mM or less, nonspecific amplification is suppressed, and a decrease in yield of a specific amplification product can be suppressed.

### 1.6. Other Components

In the case where an RNA is used as the template nucleic acid, the nucleic acid amplification reaction reagent further contains a reverse transcriptase. As the reverse transcriptase, for example, a reverse transcriptase derived from avian myeloblast virus, Ras-associated virus type 2, mouse Moloney murine leukemia virus, or human immunodefficiency virus type 1 is used.

In the case where the nucleic acid amplification reaction reagent is lyophilized, the nucleic acid amplification reaction reagent (lyophilized reagent) contains a sugar. Examples of the sugar include sucrose, trehalose, raffinose, and melezitose, each of which is a non-reducing sugar, among disaccharides and trisaccharides. Among the disaccharides and trisaccharides, particularly trehalose is preferably used because the function as a cryoprotective agent is high. Trehalose prevents the lyophilized reagent from coming into contact with a water molecule by its strong hydration force, and thus can improve the storage stability of the lyophilized reagent. The lyophilized reagent can be prepared by lyophilizing a mixed reagent solution containing the respective components of the nucleic acid amplification reaction reagent and a sugar. The temperature during lyophilization is, for example, about −80° C.

The nucleic acid amplification reaction reagent has, for example, the following characteristics.

In the nucleic acid amplification reaction reagent, the Tm value of the primer is 70° C. or higher and 80° C. or lower. Therefore, according to the nucleic acid amplification reaction reagent, in PCR, while increasing the PCR speed, amplification of a nonspecific nucleic acid (which means that a primer anneals to a region other than a target region and a nucleic acid is amplified) can be suppressed (see the below-mentioned “3. Experimental Examples” for the details). Further, in the nucleic acid amplification reaction reagent, when the nucleic acid amplification reaction reagent becomes a reaction solution for performing a nucleic acid amplification reaction, the concentration of the divalent cation contained in the reaction solution is 2 mM or more and 7.5 mM or less. Therefore, according to the nucleic acid amplification reaction reagent, while accelerating an elongation reaction by a polymerase and increasing the PCR speed, nonspecific amplification is suppressed, and a decrease in yield of a specific amplification product can be suppressed. According to the nucleic acid amplification reaction reagent, it is not necessary to use a large amount of a polymerase, and therefore, a large increase in cost for the reagent for increasing the PCR speed can be avoided.

In the nucleic acid amplification reaction reagent, when the nucleic acid amplification reaction reagent becomes a reaction solution for performing a nucleic acid amplification reaction, the concentration of MgCl₂ contained in the reaction solution may be 4 mM or more and 7.5 mM or less. Therefore, according to the nucleic acid amplification reaction reagent, while accelerating an elongation reaction by a polymerase and increasing the PCR speed, nonspecific amplification is suppressed, and a decrease in yield of a specific amplification product can be suppressed. According to the nucleic acid amplification reaction reagent, it is not necessary to use a large amount of a polymerase, and therefore, a large increase in cost for the reagent for increasing the PCR speed can be avoided.

In the nucleic acid amplification reaction reagent, when the nucleic acid amplification reaction reagent becomes a reaction solution for performing a nucleic acid amplification reaction, the concentration of MgSO₄ contained in the reaction solution may be 2 mM or more and 3 mM or less. Therefore, according to the nucleic acid amplification reaction reagent, while accelerating an elongation reaction by a polymerase and increasing the PCR speed, nonspecific amplification is suppressed, and a decrease in yield of a specific amplification product can be suppressed. According to the nucleic acid amplification reaction reagent, it is not necessary to use a large amount of a polymerase, and therefore, a large increase in cost for the reagent for increasing the PCR speed can be avoided.

In the nucleic acid amplification reaction reagent, the Tm value of the primer may be 70° C. or higher and 75° C. or lower. Therefore, according to the nucleic acid amplification reaction reagent, in PCR, while increasing the PCR speed, amplification of a nonspecific nucleic acid can be more reliably suppressed.

In the nucleic acid amplification reaction reagent, the primer may contain an artificial nucleic acid. Therefore, according to the nucleic acid amplification reaction reagent, the Tm value of the primer can be made to fall within the range of 70° C. or higher and 80° C. or lower without increasing the number of bases of the primer, and nonspecific adsorption of a primer dimer can be suppressed.

In the nucleic acid amplification reaction reagent, the probe may be a hydrolysis probe. Therefore, according to the nucleic acid amplification reaction reagent, in PCR, when the probe is degraded by the polymerase, a quenching effect is cancelled, and a reporter dye emits light, whereby the amplification amount of a nucleic acid can be quantitatively determined.

In the nucleic acid amplification reaction reagent, the probe may contain at least one of an artificial nucleic acid and a minor groove binder molecule. Therefore, according to the nucleic acid amplification reaction reagent, in PCR, the Tm value of the probe can be increased without increasing the number of bases of the probe.

The nucleic acid amplification reaction reagent may be lyophilized. According to this, the nucleic acid amplification reaction reagent can be stably stored.

### 2. Nucleic Acid Amplification Reaction Method

Next, the nucleic acid amplification reaction method according to this embodiment will be described with reference to the accompanying drawings. FIG. 2 is a flowchart for illustrating the nucleic acid amplification reaction method according to this embodiment.

First, a reaction solution is prepared by bringing the nucleic acid amplification reaction reagent according to this embodiment and a template nucleic acid solution into contact with each other (Step S1). Specifically, a template nucleic acid solution is introduced using a pipette or the like into a container in which the nucleic acid amplification reaction reagent is placed so as to bring the nucleic acid amplification reaction reagent and the template nucleic acid solution into contact with each other, whereby a reaction solution is prepared.

The template nucleic acid solution is obtained, for example, as follows. That is, a specimen, for example, a cell derived from an organism such as a human or a bacterium, a virus, or the like is collected using a collecting tool such as a cotton swab, and a template nucleic acid is extracted from the specimen using a known extraction method. Thereafter, a template nucleic acid solution is purified so as to have a predetermined concentration using a known purification method. The solution in the template nucleic acid solution is, for example, water (distilled water or sterile water) or a Tris-EDTA (ethylenediaminetetraacetic acid) (TE) solution.

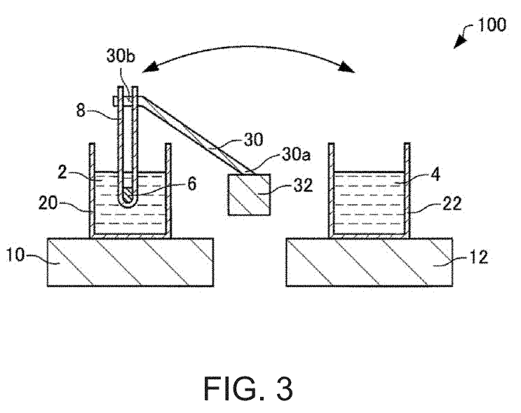

Subsequently, thermal cycling (for PCR) for amplifying a nucleic acid is performed for the reaction solution (Step S2). Here, FIG. 3 is a cross-sectional view schematically showing a thermal cycler 100 for performing thermal cycling for a reaction solution 6 according to this embodiment.

As shown in FIG. 3, the thermal cycler 100 includes a first hot plate 10, a second hot plate 12, a first beaker 20, a second beaker 22, an arm 30, and a fixing section 32.

The first hot plate 10 heats a liquid 2 contained in the first beaker 20 to a first temperature. The first temperature is a temperature suitable for the dissociation (denaturation reaction) of a double-stranded DNA, and is, for example, 85° C. or higher and 105° C. or lower. The type of the liquid 2 is not particularly limited as long as it can be heated to the first temperature by the first hot plate 10, and for example, an aqueous sodium chloride solution and an oil can be exemplified.

The second hot plate 12 heats a liquid 4 contained in the second beaker 22 to a second temperature. The second temperature is lower than the first temperature. The second temperature is a temperature suitable for an annealing reaction and an elongation reaction, and is, for example, 55° C. or higher and 75° C. or lower. That is, in this step, in the heating for the annealing reaction of the primer, the elongation reaction is performed. That is, the annealing reaction and the elongation reaction are performed at the same temperature. According to the above-mentioned FIG. 1, from the viewpoint of the activity efficiency of the polymerase, as the second temperature, around 70° C. is most suitable. The type of the liquid 4 is not particularly limited as long as it can be heated to the second temperature by the second hot plate 12, and for example, an aqueous sodium chloride solution and an oil can be exemplified.

The arm 30 is configured such that one end 30a is fixed by the fixing section 32 and the other end 30b is a free end. The end 30b of the arm 30 supports the container 8 containing the reaction solution 6. The arm 30 is operated by a motor (not shown) such that the end 30b reciprocates arcuately while fixing the end 30a.

By the reciprocation of the arm 30, the reaction solution 6 is alternately placed in the liquid 2 heated to the first temperature and in the liquid 4 heated to the second temperature. According to this, thermal cycling for PCR can be performed for the reaction solution 6. The number of cycles of the thermal cycling in this step can be appropriately set by driving and stopping of the motor, and for example, 20 or more and 60 or less. The conveying time of the reaction solution 6 from the liquid 2 to the liquid 4 and the conveying time of the reaction solution 6 from the liquid 4 to the liquid 2 are preferably as short as possible, but are, for example, about 0.5 seconds.

In the thermal cycling step (Step S2), a heating time for the denaturation reaction per cycle (in the example shown in the drawing, a time in which the reaction solution 6 is placed in the liquid 2) is, for example, 0.3 seconds or more and 5 seconds or less, preferably 0.5 seconds or more and 2 seconds or less. By setting the heating time for the denaturation reaction to 0.3 seconds or more, it is possible to suppress insufficient denaturation due to a too short denaturation reaction time. By setting the heating time for the denaturation reaction to 5 seconds or less, the PCR speed can be increased.

In the thermal cycling step (Step S2), a heating time for the annealing reaction and the elongation reaction per cycle (in the example shown in the drawing, a time in which the reaction solution 6 is placed in the liquid 4) is, for example, 6 seconds or less, preferably 4 seconds or less, more preferably 1 second or more and 3 seconds or less, further more preferably 1 second or more and 1.5 seconds or less. By setting the heating time for the annealing reaction and the elongation reaction to 6 seconds or less, the PCR speed can be increased.

In the thermal cycling step (Step S2), a time per cycle of the thermal cycling is 9 seconds or less, preferably 7 seconds or less, more preferably 6 seconds or less. By setting the time per cycle to 9 seconds or less, the thermal cycling speed can be increased. The time per cycle of the thermal cycling includes a time required for the denaturation reaction, the annealing reaction, and the elongation reaction, and the conveying time of the reaction solution for performing these reactions (for example, the conveying time of the reaction solution 6 from the liquid 2 to the liquid 4 and the conveying time of the reaction solution 6 from the liquid 4 to the liquid 2).

Subsequently, the fluorescence intensity of the reaction solution is measured (Step S3). For example, the reaction solution after thermal cycling is performed is transferred to a light transmissive container, and the fluorescence intensity is measured by irradiating the light transmissive container with light. By doing this, the amplification amount of the nucleic acid can be quantitatively determined.

In the nucleic acid amplification reaction method, in the thermal cycling step, the time per cycle of the thermal cycling is 9 seconds or less, and the Tm value of the primer is 70° C. or higher and 80° C. or lower. Therefore, according to the nucleic acid amplification reaction method, while increasing the thermal cycling speed, amplification of a nonspecific nucleic acid can be suppressed (see the below-mentioned “3. Experimental Examples” for the details).

In the nucleic acid amplification reaction method, in the heating for the annealing reaction for the primer, the elongation reaction is performed. Therefore, according to the nucleic acid amplification reaction method, the PCR speed can be increased as compared with the case where heating for the annealing reaction for the primer and heating for the elongation reaction are performed separately.

### 3. Experimental Examples

Hereinafter, the invention will be more specifically described by showing experimental examples. However, the invention is by no means limited to the following experimental examples.

### 3.1. First Experimental Example

### 3.1.1. Preparation of Reaction Solution

As a template nucleic acid (template DNA), a Mycoplasma species DNA was used. The following reaction solution was prepared by adding this template nucleic acid to a nucleic acid amplification reaction reagent.

### Composition of Reaction Solution

| | |
|---|---|
| Platinum Taq polymerase (5 units/μL) | 0.4 μL |
| Buffer | 2.0 μL |
| dNTP (10 mM) | 0.25 μL |
| Forward primer for detection of Mycoplasma species (20 μM) | 1.2 μL |
| Reverse primer for detection of Mycoplasma species (20 μM) | 1.2 μL |
| Fluorescently labeled probe for detection of Mycoplasma | 0.9 μL |
| species (10 μM) | |
| Mycoplasma species DNA (100 copies/μL) | 1.0 μL |
| Distilled water | 3.05 μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

In this experiment, primers having a different Tm value were used. Specifically, primers having a Tm value of about 60° C. (Tm60), about 70° C. (Tm70), about 75° C. (Tm75), about 80° C. (Tm80), or about 85° C. (Tm85) were used. The Tm values and the sequences of the primers, and the sequence of the probe are as shown in the following Table 1.

TABLE 1

| | | Tm (° C.) | SEQ ID NO: | Sequence |
|---|---|---|---|---|
| Tm 60 | Forward | 62.6 | 1 | 5′ AAA TCC AGG TAC GGG TGA AG 3′ |
| | primer | | | |
| | Reverse | 60.6 | 2 | 5′ GTC CTG ATC AAT ATT AAG CTA CAG TAA A 3′ |
| | primer | | | |
| Tm 70 | Forward | 70.4 | 3 | 5′ AAA TCC AGG TAC GGG TGA AGA CAC C 3′ |
| | primer | | | |
| | Reverse | 70.7 | 4 | 5′ GTC CTG ATC AAT ATT AAG CTA CAG TAA AGC TTC |
| | primer | | | ACG 3′ |
| Tm 75 | Forward | 75.9 | 5 | 5′ GGT GAA ATC CAG GTA CGG GTG AAG ACA CC 3′ |
| | primer | | | |
| | Reverse | 75.4 | 6 | 5′ GTC CTG ATC AAT ATT AAG CTA CAG TAA AGC TTC |
| | primer | | | ACG GGG 3′ |
| Tm 80 | Forward | 80.2 | 7 | 5′ GGT GAA ATC CAG GTA CGG GTG AAG ACA CCC G 3′ |
| | primer | | | |
| | Reverse | 79.0 | 8 | 5′ CAT GAT AAT GTC CTG ATC AAT ATT AAG CTA CAG |
| | primer | | | TAA AGC TTC ACG GGG TC 3′ |
| Tm 85 | Forward | 85.5 | 9 | 5′ GGT GAA ATC CAG GTA CGG GTG AAG ACA CCC GTT |
| | primer | | | AGG CGC 3′ |
| | Reverse | 84.9 | 10 | 5′ GCA TCG ATT GCT CCT ACC TAT TCT CTA CAT GAT |
| | primer | | | AAT GTC CTG ATC AAT ATT AAG CTA CAG TAA AGC TTC |
| | | | | ACG GGG TC 3′ |
| Probe | | | 11 | 5′ FAM-CGG GAC GGA AAG ACC-NFQ-MGB 3′ |

The Tm values shown in Table 1 were calculated according to the above-mentioned formula (1), and the calculation was performed by setting Ct to 500 nM and Na⁺ to 50 mM in the formula (1).

### 3.1.2. Results of Experiment

10 μL of the reaction solution as described above was placed in a container (Light Cycler Capillaries (20 μL) manufactured by Roche), and PCR was performed by allowing the container to reciprocate between a high-temperature region and a low-temperature region (see FIG. 3). The number of cycles of the thermal cycling was set to 40. Thereafter, the reaction solution was transferred to a different container (MicroAmp Fast Reaction Tubes, manufactured by Applied Biosystems, Inc.), and a fluorescence intensity was measured using a Step one Plus Real-time PCR system manufactured by Applied Biosystems, Inc.

In the PCR using each of the primers (Tm60, Tm70, Tm75, Tm80, and Tm85), the heating temperature (high temperature) for the denaturation reaction was set to 87° C. In the PCR using Tm60, Tm70, Tm75, Tm80, and Tm85, the heating temperature (low temperature) for the annealing reaction and the elongation reaction was set to 60° C., 63° C., 66° C., 69° C., and 72° C., respectively. In the PCR, the heating time (the time at the high temperature) for the denaturation reaction per cycle, the heating time (the time at the low temperature) for the annealing reaction and the elongation reaction per cycle, and the reaction time are shown in the following Table 2. Incidentally, in order to activate the polymerase, the reaction solution was initially heated to the high temperature for 10 seconds (hot start). The reaction time is obtained by, in addition to the polymerase activation time, adding the time at the high temperature and the time at the low temperature multiplied by 40 (the number of cycles), and further adding the conveying time of the reaction solution. Further, in Table 2, the time per cycle of the thermal cycling is obtained by adding the conveying time from the high-temperature region to the low-temperature region (0.5 sec) and the conveying time from the low-temperature region to the high-temperature region (0.5 sec) to the sum of the time at the high temperature and the time at the low temperature. For example, in the case where the reaction time is 370 seconds, the time per cycle of the thermal cycling is as follows: the time at the high temperature (2 sec)+the time at the low temperature (6 sec)+the conveying time from the high-temperature region to the low-temperature region (0.5 sec)+the conveying time from the low-temperature region to the high-temperature region (0.5 sec)=9 sec.

TABLE 2

| | | | | | | | |
|---|---|---|---|---|---|---|---|
| Time at high | 2 | 2 | 2 | 2 | 2 | 2 | 4 |
| temperature (sec) | | | | | | | |
| Time at low | 1 | 1.5 | 2 | 3 | 4 | 6 | 6 |
| temperature (sec) | | | | | | | |
| Reaction time (sec) | 170 | 190 | 210 | 250 | 290 | 370 | 450 |

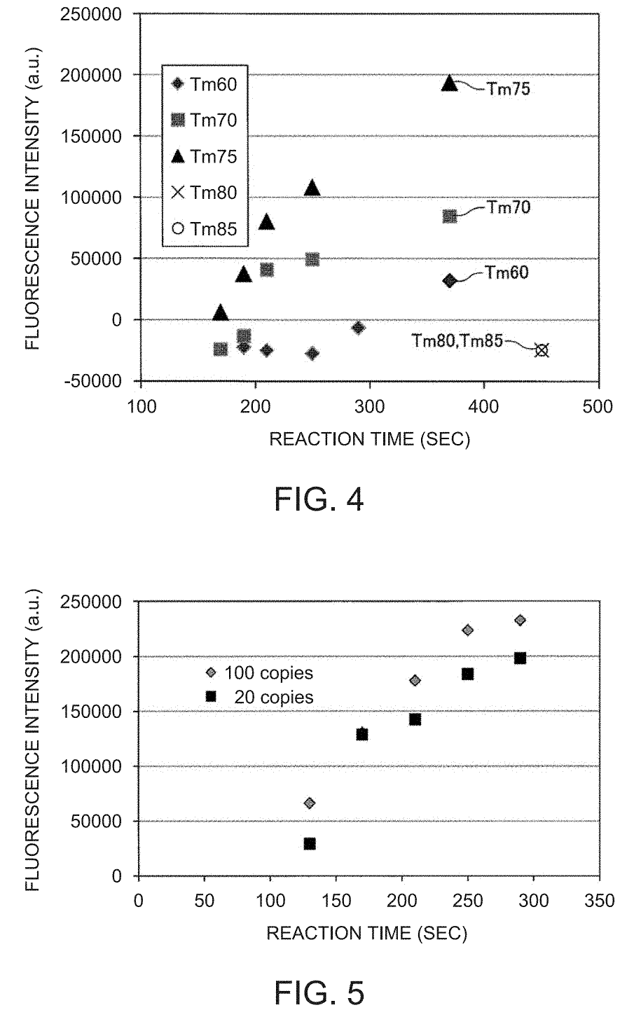

FIG. 4 is a graph showing a relationship between a PCR reaction time and a fluorescence intensity. As shown in FIG. 4, in the case where the time at the low temperature was 6 seconds (in the case where the time per cycle was 9 seconds), amplification was confirmed when using Tm60, Tm70, and Tm75, however, in the case where the time at the low temperature was 4 seconds (in the case where the time per cycle was 7 seconds) or less, amplification of a nucleic acid was not confirmed when using Tm60, and amplification was confirmed when using Tm70 and Tm75. Therefore, it was found that by setting the Tm value of the primer to 70° C. or higher and lower than 80° C., even in the case of high-speed PCR in which the time at the low temperature is 4 seconds or less, a nucleic acid can be amplified. This is considered to be because a primer having a higher Tm value anneals to a template nucleic acid faster, and therefore, Tm70 and Tm75 are more suitable for increasing the thermal cycling speed than Tm60. Further, according to the above-mentioned FIG. 1, it is considered that Tm70 and Tm75 have a higher polymerase activity efficiency than Tm60 and can accelerate the elongation reaction, and therefore, Tm70 and Tm75 are more suitable for increasing the thermal cycling speed than Tm60. When considering a variation in the device used in this experimental example, it can be said that when the fluorescence intensity is 35000 or more, a nucleic acid is reliably amplified. Therefore, in the case where the time per cycle is 9 seconds, it cannot be said that a nucleic acid is reliably amplified when using Tm60, and it can be said that a nucleic acid is reliably amplified when using Tm70 and Tm75.

Further, from FIG. 4, it was found that by setting the time at the low temperature to 2 seconds or more and 4 seconds or less, and the Tm value of the primer to 70° C. or higher and 75° C. or lower, a nucleic acid can be amplified more reliably even by high-speed PCR in which the time at the low temperature is 4 seconds or less. Further, in FIG. 4, when using Tm80 and Tm85, amplification of a nucleic acid was not confirmed. This is considered to be because the Tm value was too high, and therefore, a primer dimer or the like was formed.

In FIG. 4, a value obtained by subtracting the fluorescence intensity in a state where the nucleic acid was apparently not amplified (for example, after completion of one cycle) from the fluorescence intensity after completion of 40 cycles is plotted. The plot in which the fluorescence intensity shows a negative value is considered to be a measurement error.

### 3.2. Second Experimental Example

### 3.2.1. Preparation of Reaction Solution

As a template nucleic acid (template DNA), a Bordetella pertussis DNA was used. The following reaction solution was prepared by adding this template nucleic acid to a nucleic acid amplification reaction reagent.

### Composition of Reaction Solution

| | |
|---|---|
| Platinum Taq polymerase (5 units/μL) | 0.4 μL |
| Buffer | 2.0 μL |
| dNTP (10 mM) | 0.25 μL |
| Forward primer for detection of Bordetella pertussis (100 μM) | 0.32 μL |
| Reverse primer for detection of Bordetella pertussis (100 μM) | 0.32 μL |
| Fluorescently labeled probe for detection of Bordetella | 0.9 μL |
| pertussis (10 μM) | |
| Bordetella pertussis DNA (20 copies or 100 copies/μL) | 1.0 μL |
| Distilled water | 4.81 μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

The Tm values and the sequences of the primers, and the sequence of the probe are as shown in the following Table 3. The Tm values shown in Table 3 were calculated in the manner as the Tm values shown in Table 1.

TABLE 3

| | Tm ( C.) | SEQ ID NO: | Sequence |
|---|---|---|---|
| Forward | 80.8 | 12 | 5′ ATC AAG CAC CGC TTT ACC CGA CCT TAC CGC C 3′ |
| primer | | | |
| Reverse | 80.3 | 13 | 5′ TTG GGA GTT CTG GTA GGT GTG AGC GTA AGC CCA 3′ |
| primer | | | |
| Probe | | 14 | 5′ FAM-AAT GGC AAG GCC GAA CGC TTC A-NFQ-MGB 3′ |

### 3.2.2. Results of Experiment

PCR was performed for 10 μL of the reaction solution as described above by performing hot start for 10 seconds in the same manner as in the first experimental example. The high temperature was set to 90° C., and the low temperature was set to 60° C. The heating time (the time at the high temperature) for the denaturation reaction per cycle, the heating time (the time at the low temperature) for the annealing reaction and the elongation reaction per cycle, and the reaction time are shown in the following Table 4.

TABLE 4

| | | | | | |
|---|---|---|---|---|---|
| Time at high temperature (sec) | 1 | 2 | 2 | 2 | 2 |
| Time at low temperature (sec) | 1 | 1 | 2 | 3 | 4 |
| Reaction time (sec) | 130 | 170 | 210 | 250 | 290 |

FIG. 5 is a graph showing a relationship between a PCR reaction time and a fluorescence intensity. As shown in FIG. 5, even if the Tm value of the primer was about 80° C., amplification of a nucleic acid could be confirmed.

### 3.3. Third Experimental Example

### 3.3.1. Preparation of Reaction Solution

As a template nucleic acid (template DNA), a Mycoplasma species DNA was used. The following first reaction solution and second reaction solution were prepared by adding this template nucleic acid to a nucleic acid amplification reaction reagent.

### Composition of First Reaction Solution

| | |
|---|---|
| Platinum Taq polymerase (5 units/μL) | 0.2 μL |
| Buffer | 2.0 μL |
| dNTP (10 mM) | 0.2 μL |
| Forward primer for detection of Mycoplasma species (20 μM) | 0.4 μL |
| Reverse primer for detection of Mycoplasma species (20 μM) | 0.4 μL |
| Fluorescently labeled probe for detection of Mycoplasma | 0.2 μL |
| species (10 μM) | |
| Mycoplasma species DNA (100 copies/μL) | 1.0 μL |
| Distilled water | 5.6 μL |

### Composition of Second Reaction Solution

| | |
|---|---|
| Platinum Taq polymerase (5 units/μL) | 0.4 μL |
| Buffer | 2.0 μL |
| dNTP (10 mM) | 0.25 μL |
| Forward primer for detection of Mycoplasma species (20 μM) | 1.2 μL |
| Reverse primer for detection of Mycoplasma species (20 μM) | 1.2 μL |
| Fluorescently labeled probe for detection of Mycoplasma | 0.9 μL |
| species (10 μM) | |
| Mycoplasma species DNA (100 copies/μL) | 1.0 μL |
| Distilled water | 3.05 μL |

As the fluorescently labeled probe in both of the first reaction solution and the second reaction solution, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

In the first reaction solution, a primer having a Tm value of about 60° C. used in the first experimental example was used, and in the second reaction solution, a primer having a Tm value of about 70° C. used in the first experimental example was used (see Table 1).

In the reaction solution as described above, lysozyme was mixed as a contaminant. Specifically, lysozyme was mixed in the reaction solution so that the final concentration of lysozyme was 200 ng/μL, 20 ng/μL, or 2 ng/μL.

### 3.3.2. Results of Experiment

PCR was performed for the reaction solution as described above under a standard cycling condition using a Step one Plus Real-time PCR system manufactured by Applied Biosystems, Inc. The standard cycling condition was set as follows: the high temperature: 95° C., the time at the high temperature: 15 seconds, the low temperature: 58° C., the time at the low temperature: 30 seconds.

Further, PCR was performed for the reaction solution as described above under a high-speed cycling condition using the same device as in the first experimental example (see FIG. 3). The high-speed cycling condition was set as follows: the high temperature: 89° C., the time at the high temperature: 2 seconds, the low temperature: 65° C., the time at the low temperature: 2 seconds.

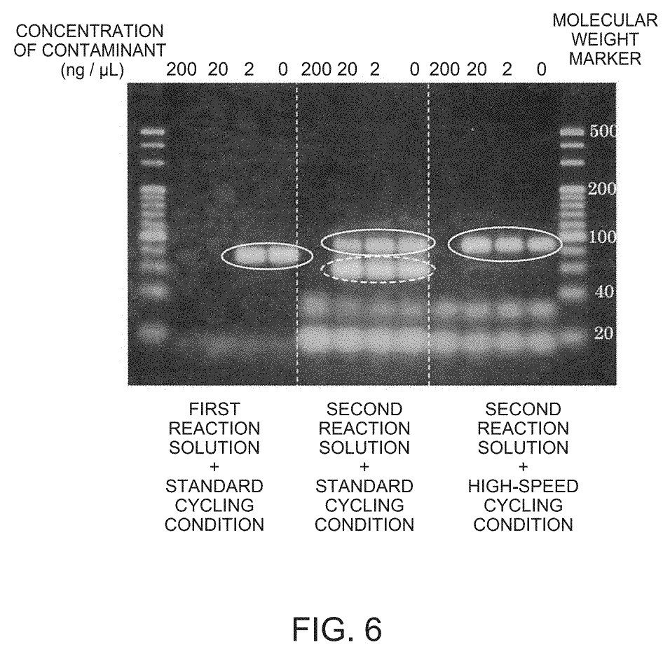

FIG. 6 shows the results of electrophoresis in the case where PCR was performed for the first reaction solution under the standard cycling condition, in the case where PCR was performed for the second reaction solution under the standard cycling condition, and in the case where PCR was performed for the second reaction solution under the high-speed cycling condition.

As shown in FIG. 6, in the case where PCR was performed for the first reaction solution under the standard cycling condition, the target band was not confirmed when the concentration of the contaminant was 20 ng/μL or more. In the case where PCR was performed for the second reaction solution under the standard cycling condition, a nonspecific band (a band other than the target band) was confirmed even when the contaminant was not mixed (the concentration of the contaminant was 0 ng/μL). In the case where PCR was performed for the second reaction solution under the high-speed cycling condition, a nonspecific band was not confirmed, and the target band was confirmed even when the concentration of the contaminant was 20 ng/μL. As a result, it was found that by setting the Tm value of the primer to 70° C. or higher and increasing the PCR speed, amplification of a nonspecific nucleic acid can be decreased.

In general, a primer having a high Tm value is likely to anneal, however, if the Tm value is high, nonspecific amplification is likely to occur, and therefore, a primer having a Tm value of about 60° C. is used. However, in this experimental example, by increasing the PCR speed, even if the Tm value was 70° C. or higher, nonspecific amplification could be decreased. In FIG. 6, the target band is surrounded by a solid line, and a nonspecific band is surrounded by a broken line.

Further, in the case where the first reaction solution was used, the target band was not confirmed when the concentration of the contaminant was 20 ng/μL or more, however, in the case where the second reaction solution was used, the target band could be confirmed when the concentration of the contaminant was 20 ng/μL or less. Therefore, it was found that by setting the Tm value of the primer to 70° C. or higher, the resistance to the contaminant was improved.

### 3.4. Fourth Experimental Example

### 3.4.1. Preparation of Reaction Solution

As a template nucleic acid (template DNA), a Mycoplasma species DNA was used. The following reaction solutions were prepared by adding this template nucleic acid to a nucleic acid amplification reaction reagent.

### Composition of First Reaction Solution (Probe Method)

| | |
|---|---|
| Platinum Taq polymerase (5 units/μL) | 0.4 μL |
| Buffer | 2.0 μL |
| dNTP (10 mM) | 0.25 μL |
| Forward primer for detection of Mycoplasma species (100 μM) | 0.32 μL |
| Reverse primer for detection of Mycoplasma species (100 μM) | 0.32 μL |
| Fluorescently labeled probe for detection of Mycoplasma | 0.9 μL |
| species (10 μM) | |
| Human genome DNA (10 ng/μL) | 1.0 μL |
| Distilled water | 4.81 μL |

### Composition of Second Reaction Solution (Intercalator Method)

| | |
|---|---|
| Platinum Taq polymerase (5 units/μL) | 0.4 μL |
| Buffer | 2.0 μL |
| dNTP (10 mM) | 0.25 μL |
| Forward primer for detection of Mycoplasma species (100 μM) | 0.32 μL |
| Reverse primer for detection of Mycoplasma species (100 μM) | 0.32 μL |
| SYBR Green (25 nM) | 0.2 μL |
| Human genome DNA (10 ng/μL) | 1.0 μL |
| Distilled water | 6.53 μL |

As the fluorescently labeled probe in the first reaction solution, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

In the first reaction solution and the second reaction solution, a primer having a Tm value of about 75° C. used in the first experimental example was used (see Table 1).

Under the positive control condition for the first reaction solution and the second reaction solution, Mycoplasma species DNA was added at 100 copies/μL of the reaction solution, and the human genome DNA was not added. Further, the reaction time condition was set as follows: the time at the high temperature: 2 seconds, the time at the low temperature: 4 seconds.

Under the negative control condition for the first reaction solution and the second reaction solution, both human genome DNA and Mycoplasma species DNA were not added. The reaction time condition was set as follows: the time at the high temperature: 2 seconds, the time at the low temperature: 4 seconds.

### 3.4.2. Results of Experiment

10 μL of the reaction solution as described above was placed in a container (Light Cycler Capillaries (20 μL) manufactured by Roche), and PCR was performed by allowing the container to reciprocate between a high-temperature region and a low-temperature region. The number of cycles of the thermal cycling was set to 40. Thereafter, the reaction solution was transferred to a different container (MicroAmp Fast Reaction Tubes, manufactured by Applied Biosystems, Inc.), and a fluorescence intensity was measured using a Step one Plus Real-time PCR system manufactured by Applied Biosystems, Inc.

Further, after measuring the fluorescence intensity, 5 μL of the amplified sample was used, and 1 μL of a 6× loading buffer was added thereto, and 6 μL of the resulting solution was loaded onto an electrophoresis gel. Then, electrophoresis was performed, and a band was confirmed using a gel imaging device.

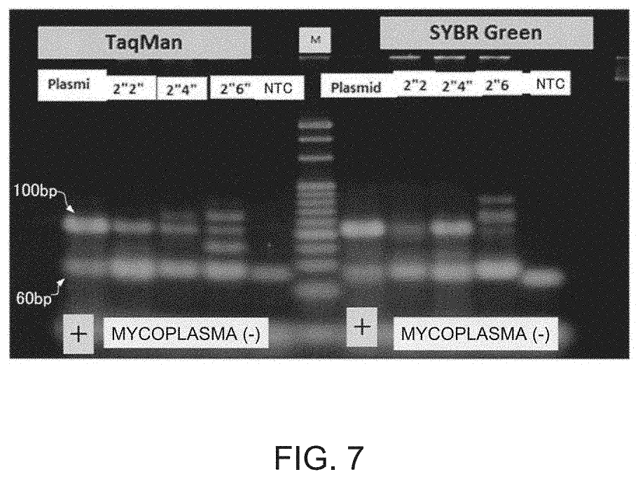

FIG. 7 shows the results of electrophoresis. In both cases of the first reaction solution using the probe and the second reaction solution using the intercalator, electrophoretic bands appear substantially similarly. In the positive control lane, a strong band appears at around 100 bp, which is the band having a target amplification length. In the negative control (NTC) lane, the target band was not confirmed, and a nonspecific amplification band having a short amplification length of around 60 bp was confirmed. Further, a band due to nonspecific amplification was confirmed in all lanes in which the reaction was confirmed by adding the human genome DNA as the contaminant (the time at the high temperature: 2 seconds/the time at the low temperature: 2 seconds to the time at the high temperature: 2 seconds/the time at the low temperature: 6 seconds). The reason why amplification was confirmed although the template DNA was not present is considered to be because in order to increase the PCR speed, a divalent cation was used at a high concentration to increase the enzymatic activity, thereby increasing the reaction efficiency, and the Tm value of the primer was increased, and therefore, nonspecific adsorption was likely to occur.

In particular, under the condition that the human genome DNA was likely to cause nonspecific adsorption, a lot of nonspecific amplification was confirmed, and although bands were separated by electrophoresis, it resulted in not being able to be distinguished whether the band is the target band or a nonspecific band. It was found that since the bands cannot be distinguished by electrophoresis in which separation is performed based on the amplification length, improvement of the specificity of the system at the time of amplification is needed instead of managing to analyze an amplification product by analysis after amplification in a low specific condition.

By decreasing the annealing time and increasing the reaction rate, nonspecific bands are reduced, and therefore, it is found that by increasing the reaction rate, nonspecific adsorption can be prevented. However, even when the time at the high temperature was set to two seconds and the time at the low temperature was set to two seconds, nonspecific adsorption could not be completely eliminated although it could be reduced.

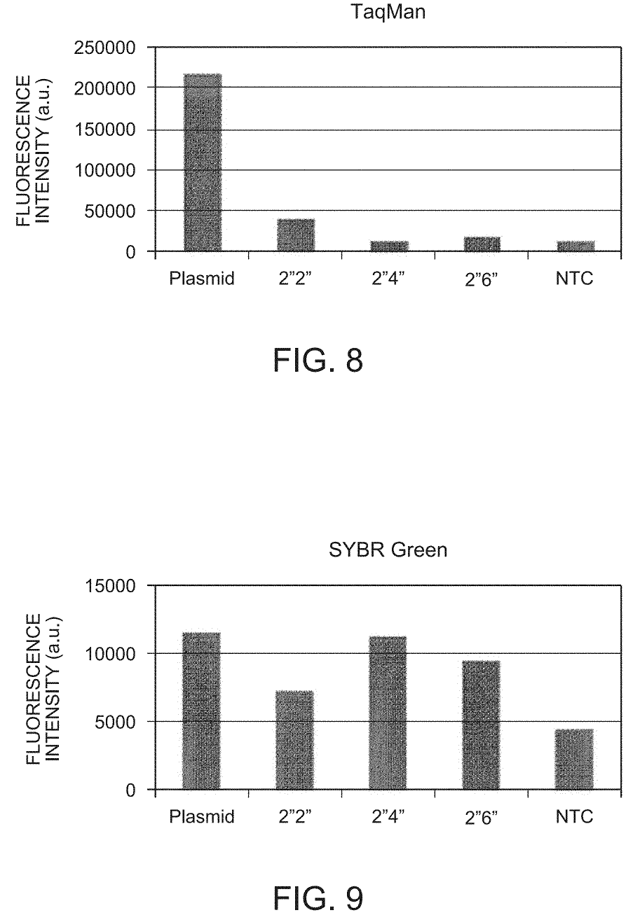

In FIG. 7, “2″2″”, “2″4″”, and “2″6″” indicate “the time at the high temperature: 2 sec/the time at the low temperature: 2 sec”, “the time at the high temperature: 2 sec/the time at the low temperature: 4 sec”, and “the time at the high temperature: 2 sec/the time at the low temperature: 6 sec”, respectively. The same applies also to the following FIGS. 8 and 9.

FIG. 8 is a graph showing the fluorescence intensity of the first reaction solution. FIG. 9 is a graph showing the fluorescence intensity of the second reaction solution. From the results of brightness variations after amplification shown in FIGS. 8 and 9, it is found that in the case of the intercalator method, a fluorescent brightness variation occurred under all the conditions, and the specificity is low, however, in the case of the probe method, a dominant fluorescent brightness variation occurred only in the positive control, and therefore, the specificity is improved by using the probe.

In order to achieve a high-speed reaction, a decrease in specificity of the reaction system cannot be avoided because priority is given to the reaction efficiency. By increasing the reaction rate, the specificity is improved, however, under the condition that nonspecific amplification is likely to occur, it is not sufficient to take only such measures. By using the probe, the specificity is improved due to the sequence selectivity of the probe, so that both high speed and high specificity of the amplification reaction system can be achieved.

The invention includes substantially the same configurations (for example, configurations having the same functions, methods, and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

The entire disclosure of Japanese Patent Application No. 2016-149490, filed Jul. 29, 2016 is expressly incorporated by reference herein.

### Sequence Listing Free Text

SEQ ID NO: 1 is the sequence of a forward primer for Mycoplasma bacteria.

SEQ ID NO: 2 is the sequence of a reverse primer for Mycoplasma bacteria.

SEQ ID NO: 3 is the sequence of a forward primer for Mycoplasma bacteria.

SEQ ID NO: 4 is the sequence of a reverse primer for Mycoplasma bacteria.

SEQ ID NO: 5 is the sequence of a forward primer for Mycoplasma bacteria.

SEQ ID NO: 6 is the sequence of a reverse primer for Mycoplasma bacteria.

SEQ ID NO: 7 is the sequence of a forward primer for Mycoplasma bacteria.

SEQ ID NO: 8 is the sequence of a reverse primer for Mycoplasma bacteria.

SEQ ID NO: 9 is the sequence of a forward primer for Mycoplasma bacteria.

SEQ ID NO: 10 is the sequence of a reverse primer for Mycoplasma bacteria.

SEQ ID NO: 11 is the sequence of a fluorescently labeled probe for Mycoplasma bacteria.

SEQ ID NO: 12 is the sequence of a forward primer for Bordetella pertussis.

SEQ ID NO: 13 is the sequence of a reverse primer for Bordetella pertussis.

SEQ ID NO: 14 is the sequence of a fluorescently labeled probe for Bordetella pertussis.

What is claimed is:

1. A nucleic acid amplification reaction method, comprising:
performing thermal cycling for amplifying a nucleic acid for a reaction solution containing a primer and a probe, wherein
in the thermal cycling, the time per cycle of the thermal cycling is 9 seconds or less, and
the Tm value of the primer is 70° C. or higher and 80° C. or lower.

2. The nucleic acid amplification reaction method according to claim 1, wherein a heating time for an annealing reaction for the primer is 6 seconds or less.

3. The nucleic acid amplification reaction method according to claim 1, wherein
the reaction solution contains a divalent cation, and
the concentration of the divalent cation contained in the reaction solution is 2 mM or more and 7.5 mM or less.

4. The nucleic acid amplification reaction method according to claim 3, wherein
the reaction solution contains MgCl₂,
the divalent cation is derived from MgCl₂, and
the concentration of MgCl₂ contained in the reaction solution is 4 mM or more and 7.5 mM or less.

5. The nucleic acid amplification reaction method according to claim 3, wherein
the reaction solution contains MgSO₄,
the divalent cation is derived from MgSO₄, and
the concentration of MgSO₄ contained in the reaction solution is 2 mM or more and 3 mM or less.

6. The nucleic acid amplification reaction method according to claim 1, wherein the Tm value of the primer is 70° C. or higher and 75° C. or lower.

7. The nucleic acid amplification reaction method according to claim 1, wherein the primer contains an artificial nucleic acid.

8. The nucleic acid amplification reaction method according to claim 1, wherein in the heating for the annealing reaction for the primer, an elongation reaction is performed.

9. The nucleic acid amplification reaction method according to claim 1, wherein the probe is a hydrolysis probe.

10. The nucleic acid amplification reaction method according to claim 1, wherein the probe contains at least one of an artificial nucleic acid and a minor groove binder molecule.

11. A nucleic acid amplification reaction reagent, which is a nucleic acid amplification reaction reagent for amplifying a nucleic acid, comprising a primer, a probe, and MgCl₂, wherein
the Tm value of the primer is 70° C. or higher and 80° C. or lower, and
when the nucleic acid amplification reaction reagent becomes a reaction solution for performing a nucleic acid amplification reaction, the concentration of MgCl₂ contained in the reaction solution is 4 mM or more and 7.5 mM or less.

12. A nucleic acid amplification reaction reagent, which is a nucleic acid amplification reaction reagent for amplifying a nucleic acid, comprising a primer, a probe, and MgSO₄, wherein
the Tm value of the primer is 70° C. or higher and 80° C. or lower, and
when the nucleic acid amplification reaction reagent becomes a reaction solution for performing a nucleic acid amplification reaction, the concentration of MgSO₄ contained in the reaction solution is 2 mM or more and 3 mM or less.

13. The nucleic acid amplification reaction reagent according to claim 11, wherein the reagent is lyophilized.
